(19) United States
(12) Plant Patent Application Publication
(10) Pub. No.: US 2014/0033376 P1
(43) Pub. Date: Jan. 30, 2014

(54) Prunus rootstock named 'AP3'

(50) ;

(71) Applicant: Gennadiy Eremin, Krasnodar Region (RU)

(72) Inventor: Gennadiy Eremin, Krasnodar Region (RU)

(73) Assignee: Krymsk Experimental Breeding Station, Krasnodar Region (RU)

(21) Appl. No.: 13/507,760

(22) Filed: Jul. 26, 2012

(51) Int. Cl. A01H 5/00

(52) U.S. Cl. PLT/180

(57) ABSTRACT

A new and distinct (Prunus pumila L.×P. salicina Lindl.)×P. cerasifera Ehrh. plant used as a rootstock for many different varieties that exhibits desirable disease resistance.

### BOTANICAL CLASSIFICATION

(Prunus pumila L.×P. salicina Lindl.)×P. cerasifera Ehrh.

### VARIETAL DENOMINATION

‘AP3’

### BACKGROUND OF THE INVENTION

The present invention comprises a new and distinct cultivar of (Prunus pumila L.×P. salicina Lindl.)×P. cerasifera Ehrh. used as a rootstock known by the varietal name ‘AP3’. The new variety was discovered in the Krasnodar region of Russia in 1999. The new variety is the result of planned breeding between a Prunus pumila×P. salicina plant named ‘Sapa’ (female parent, unpatented) and a Prunus cerasifera plant named ‘Otlichnitsa’ (male parent, unpatented). The purpose of breeding ‘AP3’ was to provide a productive and adaptive rootstock with a dwarf-like growth habit. The new variety is perfectly compatible with peach, apricot, and plum varieties. The new variety is a different species than both parents, but is a species of the Prunus genus.

When compared to Prunus rootstock ‘Marianna 4001’ (unpatented), ‘AP3’ is similar in growth habit, propagation by cuttings, and high productivity of grafted varieties. It differs from ‘Marianna 4001’ in low temperature tolerance and weak branching habit of shoots.

The new variety has been trial and field tested and has been found to retain its distinctive characteristics and remain true to type through successive propagations. The following characteristics generally distinguish ‘AP3’ from other varieties known to the breeder:

1. ‘AP3’ exhibits medium vigor;

2. Rooting can be done by cuttings or layering;

3. Thorns are not present on branches;

4. Compatible with peach, apricot, and plum varieties; and

5. Fruits of ‘AP3’ are smaller than average size, very poor and isolated.

### DESCRIPTION OF THE DRAWINGS

The accompanying photographic drawings illustrate the new variety, with the color being as nearly true as is possible with color illustrations of this type:

FIG. 1 shows a field of the new variety;

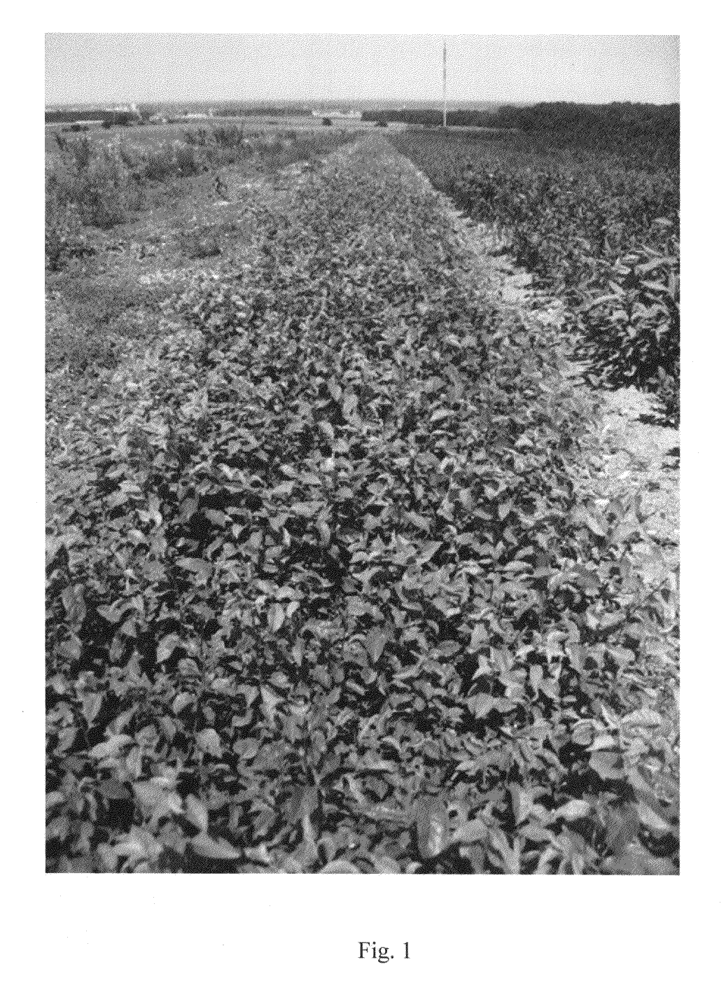

FIG. 2 shows a view of the upper surface of a leaf of the new variety; and

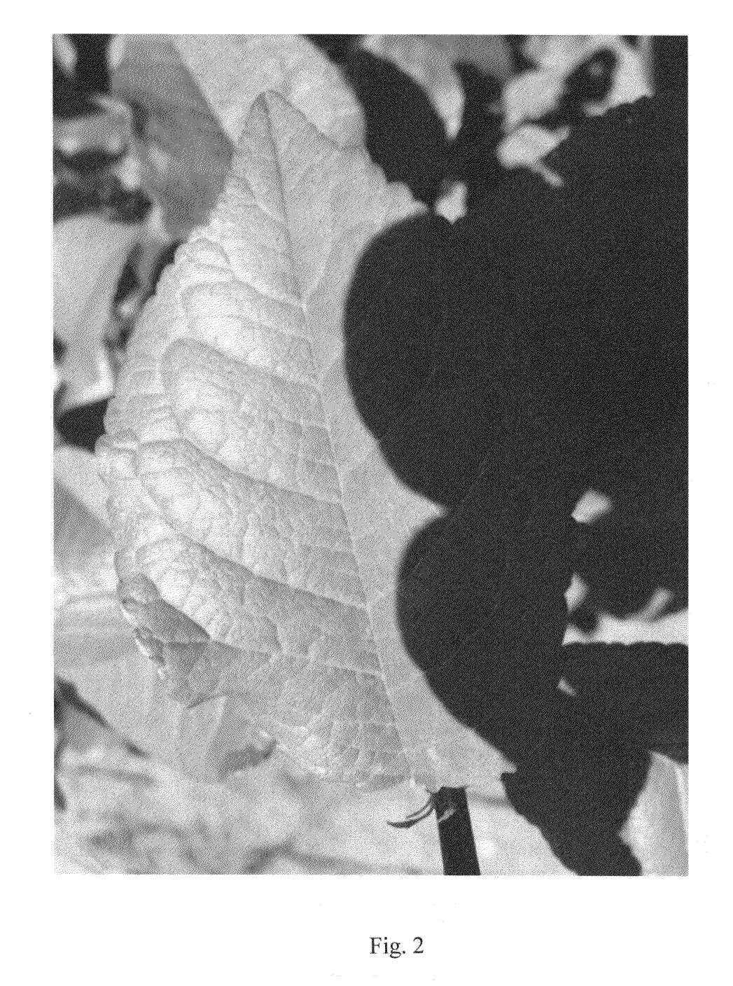

FIG. 3 shows immature plants of the new variety.

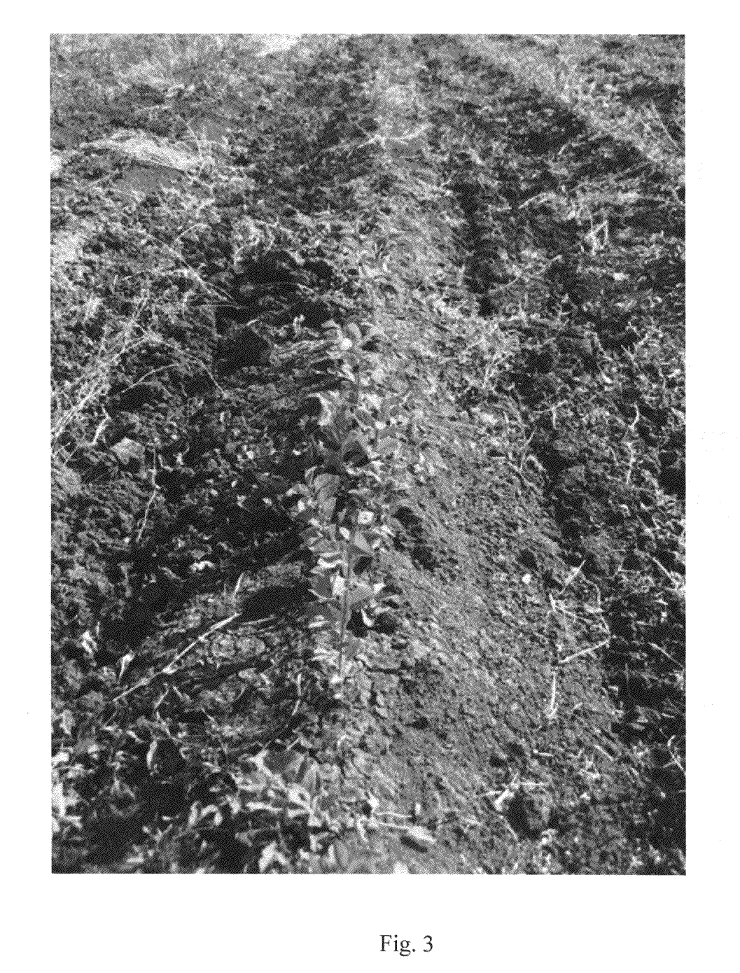

### DETAILED BOTANICAL DESCRIPTION

The following detailed description sets forth the characteristics of the new cultivar. The data which defines these characteristics were collected by asexual reproductions via cuttings and slips carried out in the Krasnodar Region of Russia. The new variety was grown in a field under warm, dry summer conditions with a temperature range of 70 to 105° F. The winter months are mild with lows to −10° F. The spring and fall months are humid. The plants were treated to regular watering and fertilizer. The color readings were taken in natural daylight. Plants were 6 years old when described.
Tree:
   Use.—Rootstock.
   Fruit bearing.—Individual fruits observed.
   Size (generally).—Medium. Height: 4.2 m. Width: 4.2 m. Density: 5×3 m.
   Average planting.—666 trees/hectare.
   Vigor.—Medium.
   Form.—Rounded.
   Growth type.—Medium.
   Trunk.—Size: 13.0 cm in diameter. Bark color: Brown. Surface texture: Smooth. Diameter: 9.0 mm at 3 years when grown in a pot. Texture: Smooth. Lenticels: Length: Average. Width: Average. Color: Brown. Density: Average.
   Branches.—Diameter: Average. Surface texture: Smooth. Color: Green. Form: Direct. Average angle: 23°. Bud arrangement: Slightly deflected. Lenticels: Length: Average. Width: Narrow. Shape: Elongated. Density: Numerous. Color: Light brown.
   Leaves.—Length: Average. Width: Average. Form: Ovoid. Texture: Average. Thickness: Thin. Base: Obtuse. Apex: Acute. Margin: Dentate-crenate. Pubescence: Light on both surfaces. Surface texture: Upper surface is glossy. Form: Narrow; elliptic. Color: Upper surface: Light brown. Lower surface: Light brown. Vein: Type: Pinnate. Color: Upper surface: Green. Lower surface: Light green. Petiole: Shape: Rounded, with shallow grooves. Length: Short. Diameter: Average. Color: Green.
   Flowers.—Buds: Length: Less than average. Width. Average; 4.0-5.0 mm. Color: Greenish-white. Pedicel: Length: 3.2 cm. Diameter: 1.0-2.0 mm. Color: Green. Bloom time: Mid-April. Pollination required: Yes. Number of flowers per raceme: Numerous. Fragrance: Weak. Petals: Number: 5 per flower. Shape: Round-oval. Length: 12.0 mm. Width: 11.0 mm. Texture: Delicate. Margin: Smooth. Aspect: Wavy. Color: Upper surface: White. Lower surface: White.
   Sepals.—Shape: Roundish. Margin: Smooth. Texture: Tight. Length: Average. Width: Average. Color: Upper surface: Green. Lower surface: Dark green with a pink tinge.
   Reproductive organs.—Anthers: Shape: Rounded. Length: Average. Color: Yellow. Stigma: Shape: Rounded. Color: Brownish-yellow. Style: Length: Average. Color: Whitish-green. Stamen number: 28. Pistil length: Stamen level. Pollen: Color: Yellow. Amount: Average.
   Fruit.—Maturity when described: Small to medium in size. Size: Diameter: 3.2 cm. Length: 3.1 cm. Skin: Thickness: Thin. Texture: Tender. Tendency to crack: None. Color: Brownish-red. Ground color: Yellow. Flesh: Aroma: None. Color: Yellowish-green. Texture: Tender. Eating quality: Satisfactory (sweet and sour). Seeds: Number per fruit: 1. Length: 2.1 cm. Breadth: 0.6 cm. Form: Rounded. Color: Brown. Stem: Length: Less than 2.0 cm. Width: Thin; 0.1 cm. Color: Green.
Hardiness and tolerance:
   Tree winter hardiness.—High.
   Bud winter hardiness.—High.
   Drought tolerance.—Higher than average.
Pathogen resistance:
   Fungal disease.—Resistant to Monilia cinerea and Clasterosporium carpophilum.
   Other diseases.—Resistant to Prunus pox virus and Root-knot nematodes.
Performance as a rootstock when grafted:
   Root sprouts (suckering).—Resistant to asphyxia.
   Anchorage.—Average.
   Compatibility.—Excellent compatibility with plum, apricot, and peach varieties.
   Vigor.—Medium; Semi-dwarf.

I claim:

1. A new and distinct variety of prunus rootstock plant, as illustrated and described herein.
